(19) United States
(12) Patent Application Publication
(10) Pub. No.: US 2016/0345789 A1
(43) Pub. Date: Dec. 1, 2016

(54) ULTRAVIOLET BACTERIA-KILLING SYSTEM FOR WATER CLOSET COVER

(71) Applicant: Gastone VITALI, Civita Castellana (VT) (IT)

(72) Inventor: Gastone VITALI, Civita Castellana (VT) (IT)

(21) Appl. No.: 15/111,354

(22) PCT Filed: Dec. 11, 2014
(86) PCT No.: PCT/IT2014/000327

§ 371 Date: Jul. 13, 2016

(30) Foreign Application Priority Data
Jan. 28, 2014 (IT) VT2014A000003

(51) Int. Cl. A47K 13/30; A61L 2/10; E03D 9/00; C02F 1/32

(52) U.S. Cl. CPC A47K 13/302; C02F 1/325; A61L 2/10; E03D 9/002; C02F 2103/005

(57) ABSTRACT

A ultraviolet bacteria-killing system for water closet cover is described, comprising: a supporting base (1); a UV lamp (2) placed on the base (1); a transparent cover (3) adapted to cover the UV lamp (2) and connected to the base (1); supply means (10) of the UV lamp (2) operatively and externally connected to the UV lamp (2); and level sensor means (6) adapted to control the switching-off of the UV lamp (2) when the water closet cover is lifted to use the water closet.

The present invention relates to an ultraviolet bacteria-killing system for water closet cover, which exploits the power of the germ-killing UV lamp to remove viruses and bacteria returning from sewages to the the syphon and the water closet surface. This system can be applied to all series of water closet covers, including those for disabled people and children.

Object of the present invention is killing bacteria from every water closet, differently from the prior art, where this did not occur.

The above and other objects and advantages of the invention, as will appear from the following description, are obtained with an ultraviolet bacteria-killing system for water closet cover as claimed in claim 1.

Preferred embodiments and non-trivial variations of the present invention are the subject matter of the dependent claims.

It is intended that the enclosed claims are an integral part of the present description.

The present invention will be better described by some preferred embodiments thereof, provided as a non-limiting example, with reference to the enclosed drawings, in which:

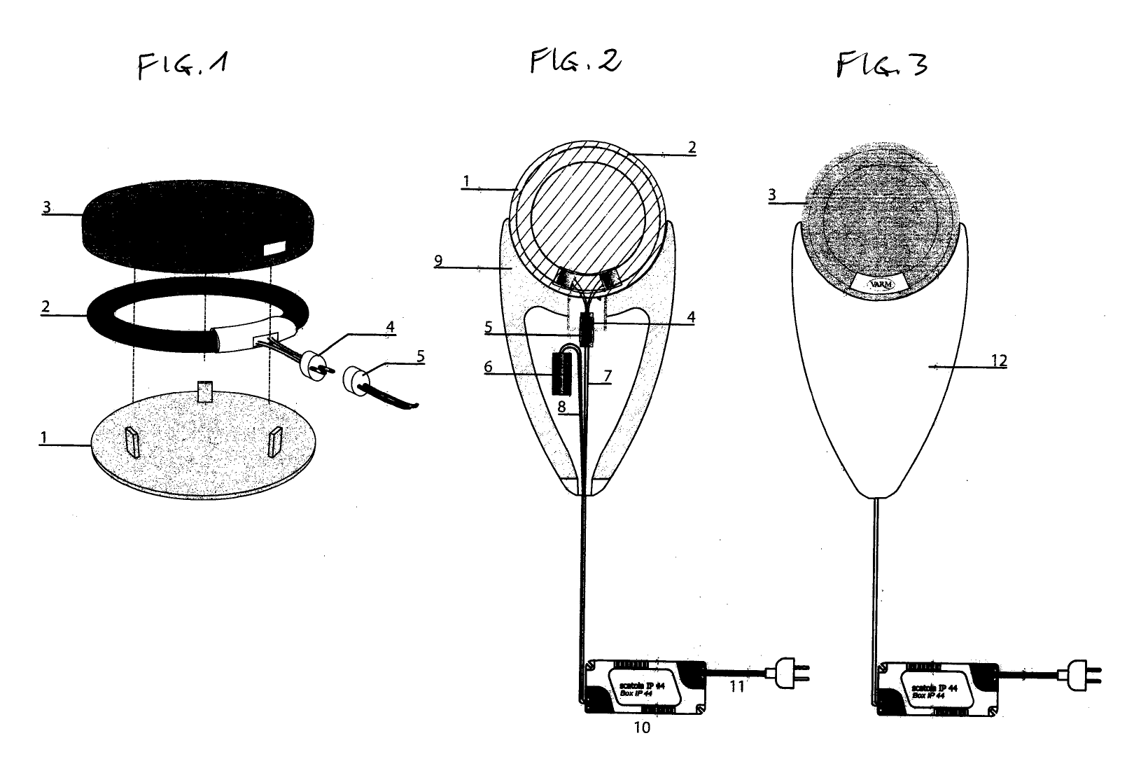

FIG. 1 shows a perspective view of the main components of the bacteria-killing system of the invention;

FIG. 2 shows a top sectional view of the system of FIG. 1 with its related supply system and applied to a water closet; and

FIG. 3 shows a top view of the system of FIG. 2.

With reference to the Figures, a preferred embodiment of the bacteria-killing device of the present invention is shown and described. It will be immediately obvious that numerous variations and modifications (for example related to shape, sizes, various colors and parts with equivalent functionalities) could be made to what is described, without departing from the scope of the invention as appears from the enclosed claims.

The invention is composed of an Ultra Violet (UV) lamp 2 having a circular shape contained inside a lamp-holder which can be applied to every series of water closet cover, which allows the complete irradiation of the surface and of the syphon of the water closet, removing viruses and bacteria coming from sewages.

The germ-killing UV lamp 2 is contained inside a lamp-holder composed of a circular base 1, preferably made of ABS, and a small dome 3, preferably made of polycarbonate, which can be extracted, arranged for being opened for replacing the lamp when it is exhausted.

The above germ-killing UV lamp 2 preferably has a circular shape with a diameter of 3 cm, 2 pins, and 5 Watts. Such arrangement of the lamp 2 allows irradiating the whole surface of the water closed and of its syphon, fighting against the return of viruses and bacteria from sewages. The mean length of the lamp 2 is 9000 hours per year, equal to a mean yearly consumption of about 6-8 Euros. The lamp 2 can reach a maximum calory of 26 C°, therefore not harmful for a human being.

The application of the above system, composed as described, is performed by means of a 2-pin male connector 4 and a 2-pin female connector 5, and a postioning sensor 6, preferably of the mercury type, which allows switching the lamp 2 off when the water closet cover is lifted for its use. This to guarantee the maximum hygiene to any bathroom user.

The cabling wires 7, 8 of the whole system are covered with a wire-covering carter 12, preferably made of thermosetting resin, which can be fastened to any seat by means of a bi-adhesive tape 9. The whole system is supplied by a 220V DC, 12V AC transformer 10, 11, having a low consumption, contained inside a small box, preferably made of ABS and IP44 certified.

The system of the present invention has been subjected to numerous analyses and experiments, and has been certified by Accredia credited Laboratories for the removal of viruses and bacteria coming from sewages and which return through the syphon till the water closet surface.

The results of the latest experiments performed on the bacteria-killing system of the present invention for water closet covers demonstrate the following:

The bacterial charge present on the water closet syphon is approximately equal to:

| | |
|---|---|
| Microbic Charge Count | 220,000 UFC/ml |
| Escherichia Coli | 74,000 UFC/ml |
| Intestine Enterococcus | 40,000 UFC/ml |

After one hour of treatment with the bacteria-killing systems of the present invention, bad smells were removed and 99% of viruses and bacteria were killed.

1-5. (canceled)

6. An ultraviolet bacteria-killing system adapted to be operatively connected to a water closet cover, the system comprising:
at least one supporting base;
at least one UV lamp (2) placed on the supporting base;
at least one transparent cover adapted to cover the UV lamp and connected to the supporting base;
supply means of the UV lamp operatively and externally connected to the UV lamp; and
level sensor means adapted to control a switching-off of the UV lamp when the water closet cover is lifted to use the water closet;
wherein:
the supply means of the UV lamp are operatively and externally connected to the UV lamp through male/female connectors capable of being detached and first wires;
the level sensor means are supplied by the supply means through second wires; and
the male/female connectors capable of being detached, a section of the first wires, a section of the second wires and the level sensor means are contained inside a covering and protecting element adapted to be placed, together with the universal bacteria-killing system, on the side of the water closet cover oriented towards inside the water closet and towards the water to be subjected to bacteria killing.

7. The system of claim 6, wherein the transparent cover is adapted to be opened for replacing the UV lamp when it is exhausted.

8. The system of claim 6, wherein the supporting base, the at least one UV lamp and the transparent cover are of a circular shape.

9. The system of claim 6, wherein the level sensor is of a mercury type.

10. The system of claim 6, wherein the supply means are composed of a low-consumption, 220V DC, 12V AC transformer contained inside a IP44 small box.
